(19) United States
(12) Patent Application Publication
(10) Pub. No.: US 2017/0281150 A1
(43) Pub. Date: Oct. 5, 2017

(54) BONE AND JOINT STABILIZATION DEVICE FEATURES

(71) Applicant: PANTHER ORTHOPEDICS, INC., San Jose, CA (US)

(72) Inventors: Kathryn A. Stecco, San Jose, CA (US); Frank P. Becking, Los Gatos, CA (US)

(21) Appl. No.: 15/473,050

(22) Filed: Mar. 29, 2017

Related U.S. Application Data
(60) Provisional application No. 62/318,037, filed on Apr. 4, 2016.

(51) Int. Cl. A61B 17/04

(52) U.S. Cl. CPC A61B 17/0401; A61B 2017/0404; A61B 2017/0403; A61B 2017/0438; A61B 2017/0445

(57) ABSTRACT

Components and associated methods of manufacture for bone and joint stabilization devices or systems are described in which medical devices are provided that include a rotatable foot as a distal anchor. The devices are tensioned during a medical procedure after applying a proximal anchor or anchoring head and remain active under tension for continued compression of the anatomy to be treated.

### CROSS-REFERENCE TO RELATED APPLICATIONS

This application claims the benefit of and priority to U.S. Provisional Application No. 62/318,037, filed Apr. 4, 2016, which is incorporated by reference herein in its entirety for all purposes.

### FIELD

The embodiments described herein are related in the field of surgery and, more particularly, for use in bone fusion, joint stabilization and/or fracture fixation surgery.

### BACKGROUND

Various devices have been employed in orthopedic surgery for bone fusion and/or joint stabilization. Bone screws, staples and plates have served as a set of rigid options. Per U.S. Pat. Nos. 4,959,064; 6,656,184; 7,833,256; 7,985,222; 8,048,134; 8,449,574 and 8,491,583 and U.S. Patent Publ. Nos. 2006/0264954 some screw-type devices have incorporated tensioning springs or members. Button-and-suture type devices have provided a more flexible set of options. U.S. Pat. Nos. 7,235,091, 7,875,057 and 8,348,960 offer examples of such device and suitable applications therefor. The subject embodiments address many shortcomings of existing products as may be appreciated by those with skill in the art.

### SUMMARY

Bone and/or joint stabilization devices are described that are advantageously tensioned during a medical procedure to remain active in maintaining compression of the subject anatomy during use. In the subject embodiments, an orthopedic surgery device or system comprises an elongate member or body, optionally comprising a spring pattern defined by a plurality of beams, each including a lateral component free to deflect when stretching the elongate body axially. An anchoring head typically receives the elongate body and may secure it with a one-way (e.g., ratcheting) interface at one end. A deployable foot is used to anchor an opposite end of the elongate body.

The anchor or anchoring foot may comprise a body with an oval, race-track or rectangular planform shape. Generally, the height, length and width of the foot will be minimized, while still maintaining adequate surface area and strength for load bearing.

Embodiments hereof are focused on features associated with this anchoring foot and optionally associated components. Optional features of the elongate body and/or anchoring heads that may be employed in the subject devices or systems are disclosed in U.S. patent application Ser. No. 15/002,022 and PCT Patent Appl. No. PCT/US16/14125 each commonly assigned, field Jan. 20, 2016 and incorporated by reference in their entireties for such description and/or any other purpose.

The foot is secured to the elongate body by one or more strand(s) of material (e.g., multi-filament suture or cord). With the strand(s) received through an aperture adjacent to a distal end of the elongate body, sections or portions of the strand are looped over proximal and distal ends of the foot to secure its position. So-connected or affixed to the elongate member, it can rotate from a position aligned with the elongate body to a position transverse (or at least angled, typically upwards of about 45 or about 60 degrees up to 90 degrees) to the elongate body for anchoring the overall device during a medical procedure.

Lateral and longitudinal movement of the foot relative to the elongate body is controlled (i.e., minimized or effectively eliminated) by virtue of sections of the strand(s) located on each side of the elongate body and optionally received within grooves in the foot. When the strands are tightened during assembly and so-fixed, the relative positioning of elements constrains other degrees of freedom, except for rotation of the foot about a horizontal axis defined by the distal end of the elongate body.

Such construction requires no pivot hinge in defining a stable rotatable interface between the elements. The interaction between the elongate body and foot with the strand portions do so instead. Nevertheless, other complicating features (such as providing a bias towards a/the transverse position by an integral or a supplemental spring to aid transition from the foot's axial delivery configuration to its implanted position) may be provided. Alternatively, one or more pull wires or cords may be employed to accomplish or assist with such rotation.

With a device setup having its foot in a body-aligned or stowed configuration, the foot and body can be inserted together through a minimum-diameter hole or channel spanning bone(s), joint space and/or a fracture. Then the system is secured or stabilized with the foot in a fully or partially transverse foot orientation, with the elongate body received in an anchoring head or otherwise clamped.

The elongate body may be covered by a sheath prior to deployment. The sheath helps to prevent tissue ingrowth once implanted. Alternatively, a/the sheath may be used to support the elongate for advancement into place and/or hold distal anchor (i.e., foot) position. The sheath may be trimmed to desired length before or after any such activity, or it may be selected from a panel of different length pre-trimmed sheaths. It may be removed as part of an overall orthopedic injury treatment method along with the elongate member and head and foot anchors, after healing. Or the sheath may be left in place, serving the purpose of allowing removal of the elongate member as part of this or these method(s), or as a separate removal procedure method.

In a method of manufacture, the foot is attached to the elongate body with one strand as detailed below or with a plurality of strands. The assembled parts may be provided in packaged combination in a kit to be acquired. They may be loaded in a sheath to preset foot position in alignment with the elongate body for implantation. In producing a final assembly (e.g., carried out by a physician in situ), the body may be received at a proximal end by an anchoring head. A tooth or multiple teeth in the anchoring head may be engaged with the elongate body and advanced relative to the body until it is stretched to a desired tension. Tooth engagement may be with through-holes in the elongate body. Finally, the elongate body may be trimmed to length with flush cutters or a customized unit—in either a method of use and/or manufacture of a final implant configuration.

In sum, the subject device or systems, kits in which they are included (with or without assembly), methods of use (e.g., implantation, during treatment of a patient while mending and/or for system removal) and manufacture (including assembly of the various components—as applicable—in vivo or ex vivo) are all included within the scope of the present disclosure.

### BRIEF DESCRIPTION OF THE DRAWINGS

The details of the subject matter set forth herein, both as to its structure and operation, may be apparent by study of the accompanying figures, in which like reference numerals may refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. Nevertheless, to-scale features (e.g., as from engineering drawings and/or photographs) may be relied upon as antecedent basis for claim support.

FIGS. 1A and 1B are front and side views of an elongate body optionally employed in the subject devices, systems and/or methods hereof.

FIGS. 2A and 2B are top and bottom perspective views of an anchoring foot for use with the elongate body in FIGS. 1A and 1B or another elongate member.

FIGS. 3A-3C are top, side-sectional and bottom views of an anchoring head that may be used with the elongate body in FIGS. 1A and 1B.

FIG. 4 is a side view of the subject foot secured to the elongate body with a looped strand of suture material.

FIGS. 5A and 5B are top and bottom views of the foot as attached in FIG. 4 with the foot rotated into alignment with the elongate body.

FIG. 6 is a perspective view of the device configuration shown in FIGS. 5A and 5B, further including a delivery sheath.

FIGS. 7A-7G are construction views detailing suture splice and loop production as well as foot attachment to the elongate body.

FIGS. 8A and 8B are opposite-facing views of the subject device or system being deployed and after deployment in performing syndesmosis repair.

### DETAILED DESCRIPTION

Various example embodiments are shown in the figures and further described below. Reference is made to these examples in a non-limiting sense, as it should be noted that they are provided to illustrate more broadly applicable aspects of the devices, systems and/or methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present invention. All such modifications are intended to be within the scope of the claims that can be made herein.

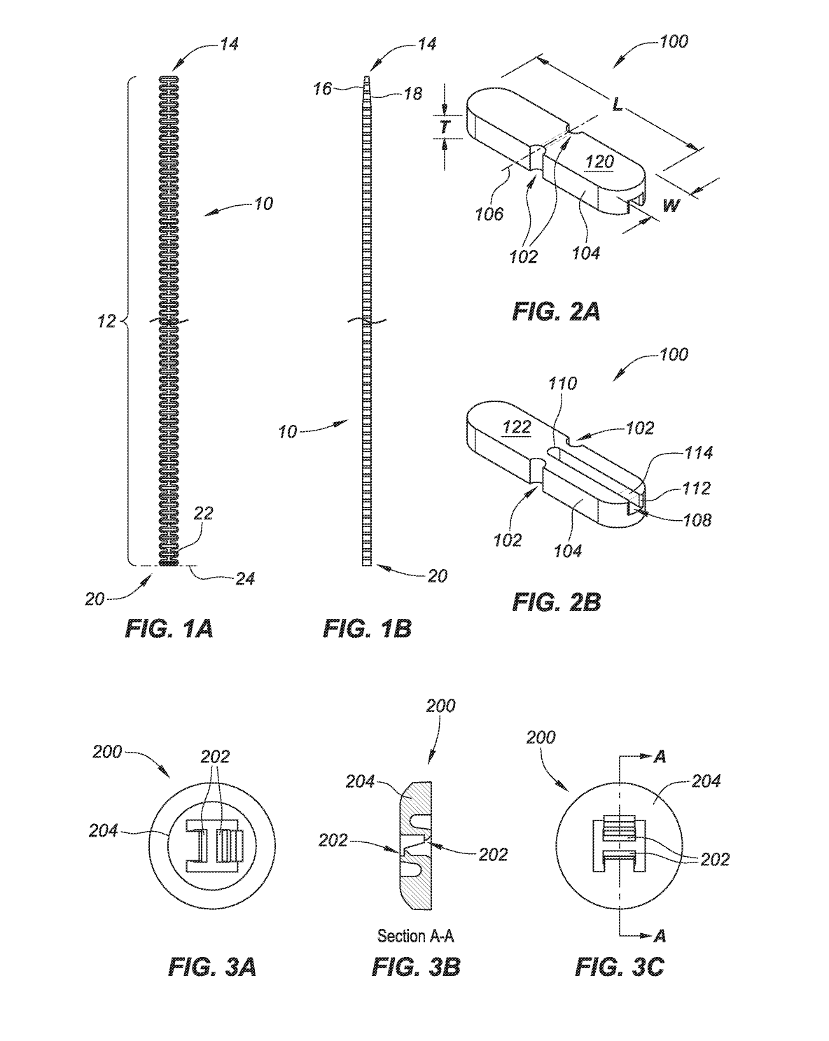

FIGS. 1A and 1B illustrate an example of an elongate body 10 to which the subject foot may be attached. In the front or plan view of FIG. 1A, an optional stretchable or spring-type architecture is evident from the repetition of laterally deflectable beams 12. The beams (or beam pairs) serve as leaf spring elements in series. As noted above, further details of such an elongate body or member 10 may be appreciated in reference to U.S. patent application Ser. No. 15/002,022 and PCT Patent Appl. No. PCT/US16/14125.

As shown in FIG. 1B, a proximal side or end 14 of elongate body 10 may be tapered to assist in loading an anchor head onto the elongate body 10. The taper is optionally defined by matching angular grinds 16, 18 or produced by other means of material removal or omission during manufacture.

The opposite, distal side 20 of the elongate body may be squared-off or (at least substantially) flat-edged as shown. Otherwise, this end may be rounded. In any case, distal end 20 provides a pivot axis 24 (roughly as indicated) about which the foot, when attached, can pivot or rotate as further described.

One or more through holes or apertures are advantageously provided adjacent to the distal end 20 of elongate body 10 for use in foot attachment. In the embodiment of body 10 shown, aperture(s) 22 that may be so-used are integrated in the spring pattern. However, the aperture(s) provided for foot attachment may instead be provided by dedicated features (vs. multi-purpose features as in elongate body 10).

FIGS. 2A and 2B provide perspective views of an anchor or anchoring foot 100 for use with elongate body 10 or another elongate member. The distal end 20 of the elongate body is intended to (at least substantially) span a proximal side 120 or surface of the foot where indicated by the phantom-lines. This location is set between diametrically-opposed side grooves or channels 102 in the sides 104 of the foot, optionally at a midpoint of the foot. Stated otherwise, the proximal end 20 of body 10 can be positioned so that a midline 106 of the length “L” of the foot and axis 24 coincide.

As illustrated and discussed further below, the grooves 102 (running or spanning the thickness “T” of the foot) receive one or more strands of material for holding or securing foot 100 to elongate body 10. Half-cylinder grooves (with edges at the full width “W” of the part) are shown for this purpose. However, V- or rectangular-shaped grooves can be used. In any case, these features offer a stable seat or seating position for the strand(s) of material used to hold the foot onto the elongate body.

The reverse (i.e., distal) side 122 of the foot may also include a recess in the form of a slot, channel or groove 108. It too is configured to receive a part of the strand(s). In a preferred embodiment, a splice section of a single loop of suture material is received therein. This approach offers an extremely low-profile solution to construction.

The distal side groove 108 may terminate adjacent to or in alignment with the position of side grooves 102. The position of an inboard terminus 110 to groove 108 will typically be set in coordination with the tie-on approach employed with the strand(s). Examples of such construction are elaborated upon below. An outboard terminus 112 of groove 108 may be open and optionally radiused (as shown) or closed-off as indicated by dashed line section 114.

An anchor or anchoring head may be used to secure elongate body 10 opposite the anchor or anchoring foot 100 in use. FIGS. 3A-3C show views of a suitable head 200 with teeth 202 designed for one-way advancement over the body. The teeth interact with the apertures or windows 22 in the body (spring) pattern shown. As illustrated with included draft angles, the anchor is advantageously injection molded in biocompatible polyetheretherketone (PEEK) polymer material. Nevertheless, other anchor and/or coordinated body configurations or constructions may be employed in the subject devices or systems.

A support rim or frame 204 of the anchor head may be round (as shown in FIGS. 3A and 3C), square or otherwise configured. Indeed, the support structure may be integrated in an orthopedic plate (e.g., as integrally formed or press-fit therein) or otherwise provided.

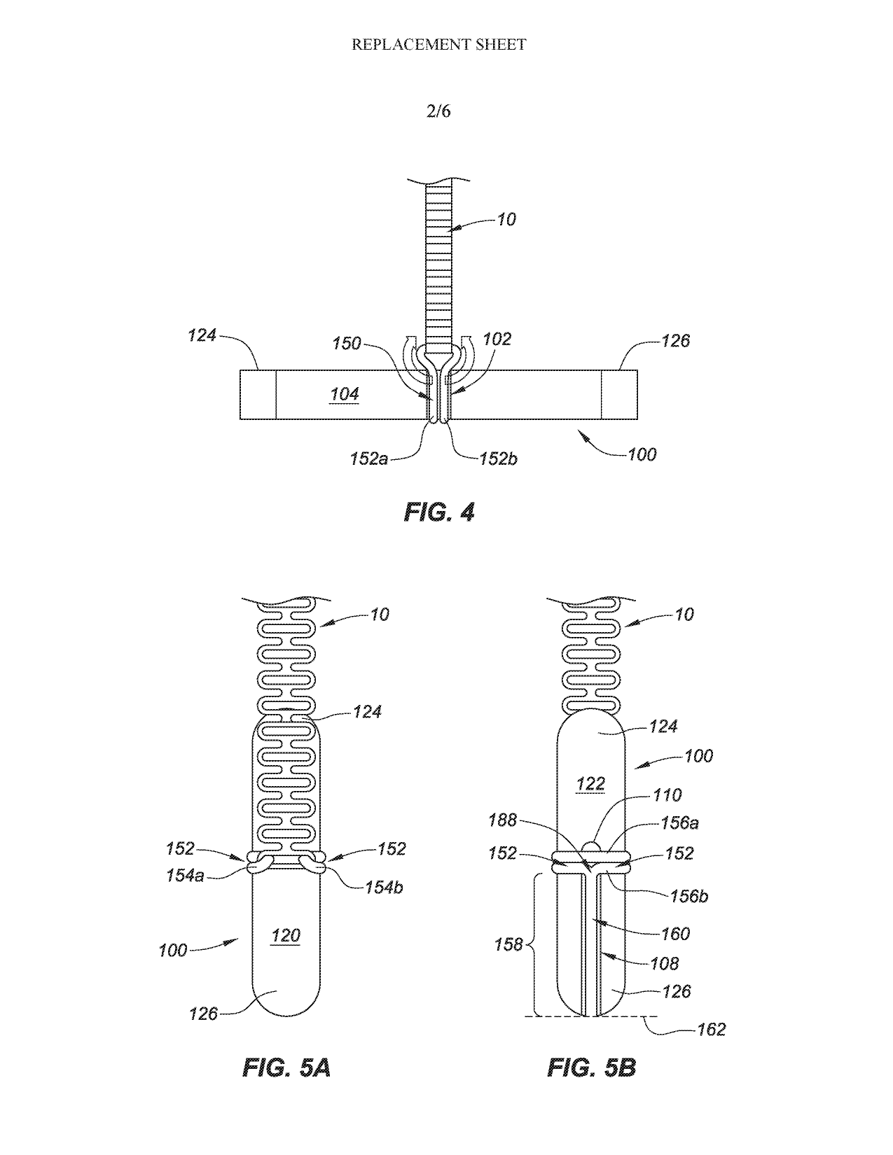

In the subject devices and/or systems, the foot 100 may be held to the elongate body with a strand 150 of suture material as shown in FIG. 4. In this example, the suture is passed and looped or wrapped over both ends 124, 126 of the foot and through aperture 22 in the elongate body 10 to provide such attachment. As such, strand 150 held in an inverted U-like or stirrup-type shape on each side of the foot.

Position of the foot relative to the elongate body is stabilized laterally by the strand given its position within the respective side groves 102. As shown, the grooves may be shaped and sized such that strand portions 152a, 152b set in a side-by-side configuration fill the available space. The foot can pivot in either direction (e.g., about the distal end 20 of the elongate body 10) and indicated by the arrows. Other motion is limited or constrained.

The nature of movement limitation or constraint is further appreciated in reference to FIG. 5A. Lateral and longitudinal constraint of the foot is applied by virtue of the loop sections 154a, 154b of the strand(s) received on each side of aperture 22, along with the portions 152 in the side grooves 102. FIG. 5B illustrates the matter further, showing how each loop portion 156a, 156b is indeed tightly and symmetrically bound in the system.

In the example pictured, a single loop of strand material is provided with one portion fed over each end 24, 26 of the length of the foot. Further, each end of the loop is secured (relative to the other) within a splice section 158. The splice section is received within the distal side or face groove 108. Splice 158 is shown adhered in place by adhesive 160 (e.g., 4014 LOCTITE). Gluing can also help insure integrity or strength of the splice against loosening and/or pull-out failure.

Alternatively, no adhesive is used and the “tail” (in this case defined by the splice) is received and held by a press or friction fit in groove 108. Regardless, the splice is typically trimmed (e.g., at dashed line 162) so that it does not extend beyond an end limit or boundary of the foot 100.

Such location for any tail features (i.e., within groove 108) and/or trimming will typically also occur even if no splice is present in the system. In which case (still using the side grooves 102), one or more knots may be used for securing one or more independent strands for tying the foot onto the elongate member.

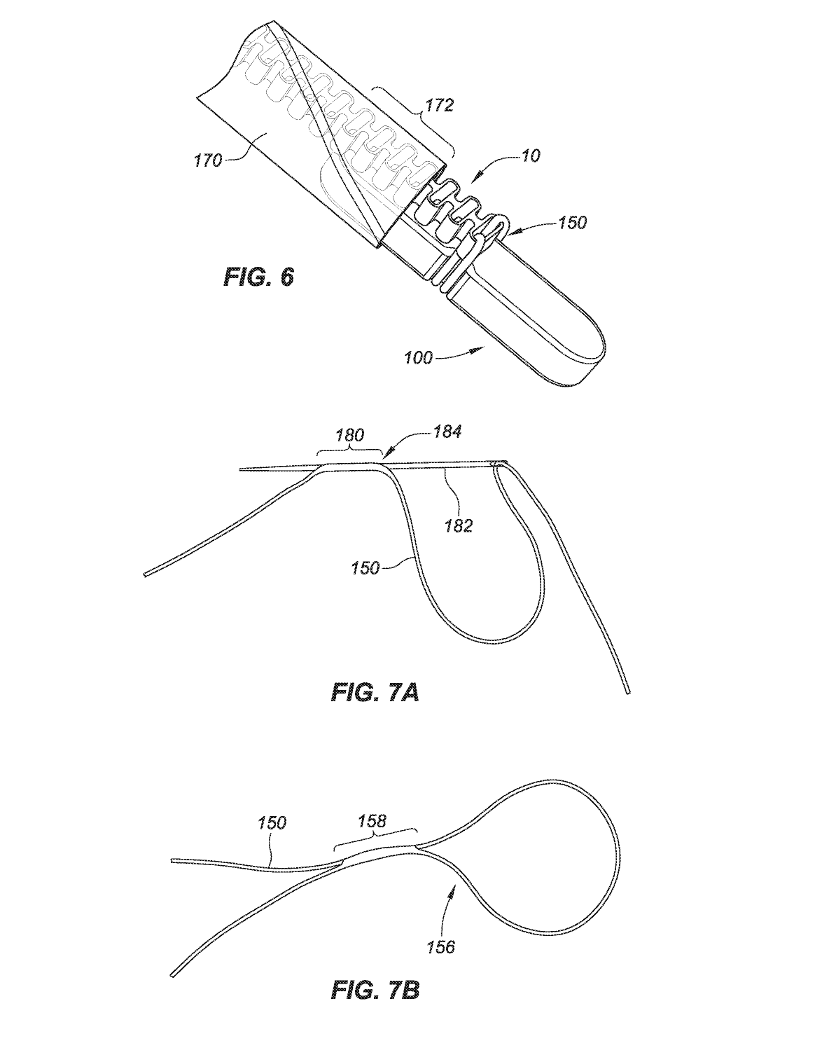

As shown in FIG. 6, the foot 100 can be releasably held or stabilized in an axial orientation for deployment in connection or association with a sleeve or sheath 170. In these figures, a clear polyester (e.g., polyethylene terephthalate (PET)) sheath is shown. However, the sheath may be any other biocompatible material. Thin-walled plastic tubing may be preferred, however, as it can easily deform from round to provide a minimum form-fitting profile or coverage 172 to the body 10 and foot 100.

As referenced above, the foot is rotatably affixed, held or secured to the elongate body by a strand(s), filament(s) or cord(s) of material. As an example, braided FORCE FIBER (Teleflex Medical) suture produced with ultra high molecular weight polyethylene (UHMWPE) material may be used. At approximately 0.010 inch diameter, 3-0 size suture material offers 15+ pound force (lfb) tensile strength for construction.

The elongate body advantageously comprises NiTi alloy that is superelastic in use at human body temperature (i.e., the material has an Af of about 37° C. or less). Otherwise, the NiTi alloy may be selected in order to use its potential shape-memory effect for tightening (or tighten further) once emplaced. Alternatively, the elongate member may comprise a high performance or so-called “engineering” polymer such as PEEK. Other materials (especially those with high reversible stain potential such as Beta titanium alloy) might be employed for elongate body 10 as well.

The elongate body is advantageously substantially flat, with the foot rotatable around its end as variously shown and described. As such, the body may have an aspect ratio of width to thickness of between about 10 to 1 and about 30 to 1. Such a form factor also minimizes manufacturing complexity and cost in that the elongate member may then be cut (e.g., laser cut, water jet cut or etched) from flat wire, strip or plate. The cut part can be media blasted, pickled and/or electropolished for surface finish.

The foot may comprise stainless steel, titanium or titanium alloy (including NiTi alloy) and be produced using standard machining or rapid-prototyping techniques. Alternatively, the foot may comprise a biocompatible polymer material such as PEEK or another suitable high-strength polymer.

Such plastic bodies are advantageously produced by injection molding with draft angles readily applied to the part. Also, the foot shown includes no through-holes (e.g., for threading suture material there-though as with suture-button type device components) otherwise complicating mold tooling. Rather, the part has side groves or slots for receiving the strand(s) that secure the foot and locate its position relative to the elongate member to which it is connected.

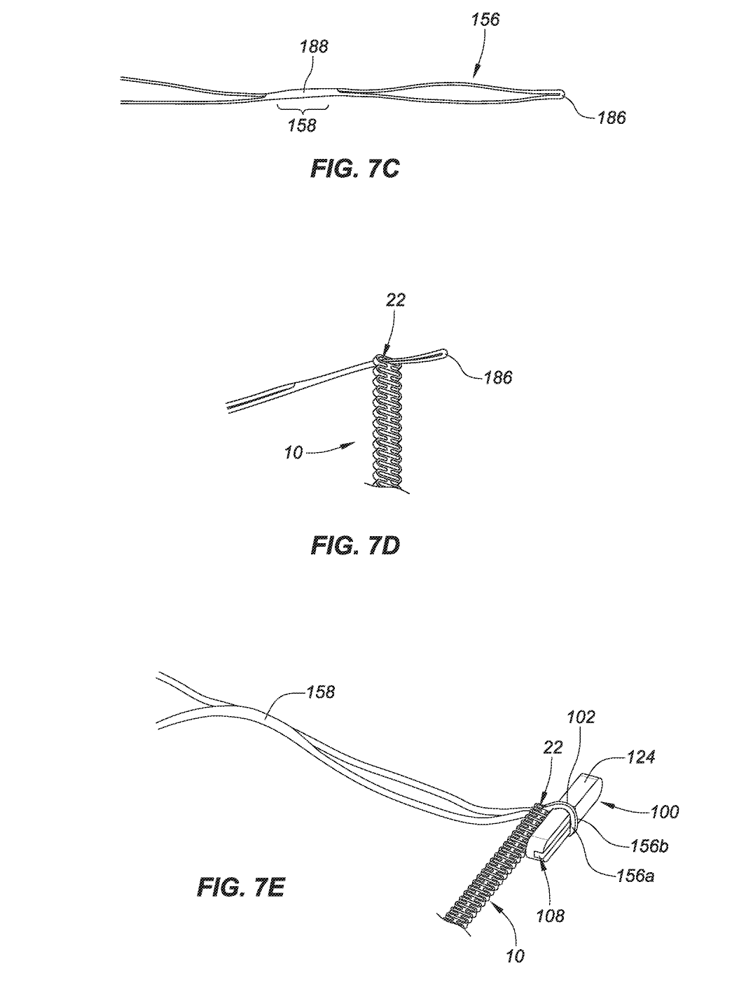

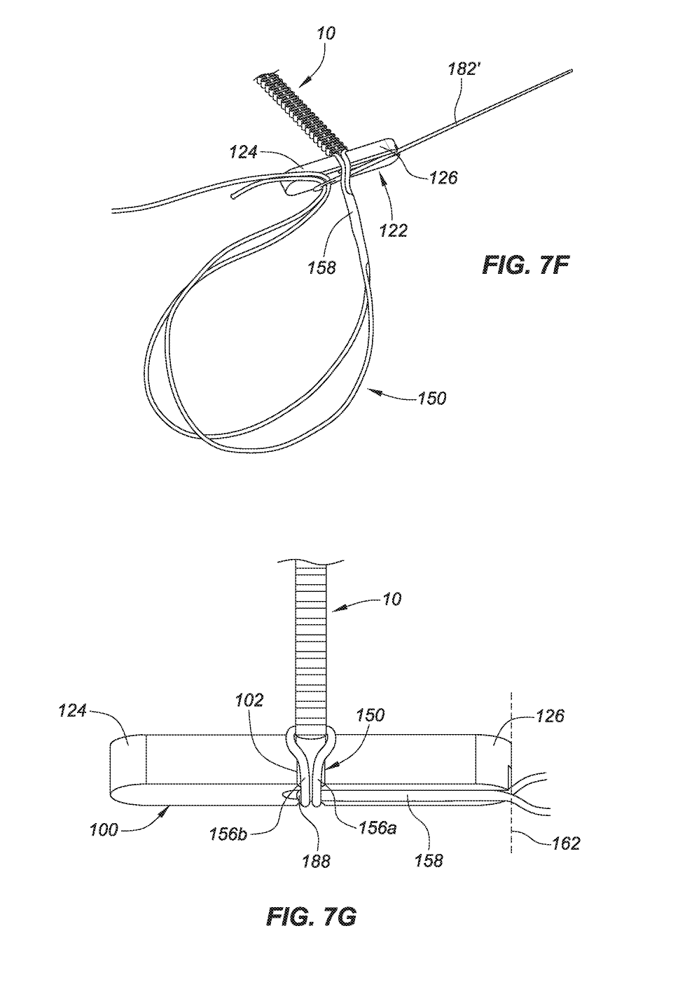

FIGS. 7A-7G illustrate a method of manufacture employing such an approach. As shown in FIG. 7A, a strand 150 comprising 6-to-8 filament hollow core suture material is opened over a splicing length 180 with a threaded needle 182. Upon passing the needle through the opening 184, a loop 156 including a splice section 158 is formed as shown in FIG. 7B.

Finished splice length may be between about 5 and 10 mm. An example of a coordinated size for the foot may be: a length of about 12.5 mm, a width of about 3 mm and a thickness of about 1.5 mm. The shape or form of foot 100 may be generically or generally regarded as oval and/or rectangular. The specific shape or form pictured is sometimes specifically referred to as a “racetrack” shape.

Turning to FIG. 7C, an end of the (straightened-out or elongated) loop 156 can be flattened (e.g., between fingers or with a tool). The flattened section 186 is then easily slipped through a low-profile aperture 22 of the body 10 (or another opening) as illustrated in FIG. 7D.

FIG. 7E shows loop 156 with one end 156a passed over and around end 126 of the foot and another end 156b passed over end 124 of foot 100. However, the overall loop 156 has not yet been tightened. This is evident in the figured from the location of the spice 158 and the offset position of the foot 100 relative to the body 10.

To tighten the loop 156, portions of the loop strand are worked through the aperture 22 and along the foot side grooves 102 until loop sections 156a and 156b are tight and the splice 158 centrally located adjacent the distal surface 122 of the foot.

Such a result in shown in FIG. 7F. Here, the offset between the parts shown in FIG. 7E is eliminated and the parts (i.e., body 10, foot 100 and strand 150 elements) are tightly held together.

If the splice is located as shown in FIG. 7F (i.e., toward foot end 124 with the distal side groove 108 and foot end 126 to the right), the strand ends may be threaded through (an optionally larger) needle 182′ and the needle used to draw the splice “tail” underneath the adjacent strand loop section 156a so that it underlies the same. So-positioned, the tail is tucked into the groove as shown in FIG. 7G, with the junction 188 of the strand sections within splice 158 neatly hidden.

Note, however, the reverse arrangement of loops 156a and 156b in FIG. 5B. Here, the splice 158 tail is originally located toward the side of groove 108 (i.e., without crossing under an adjacent strand section) facilitating omission of the acts or steps illustrated in FIG. 7F. However accomplished, with the remaining end(s) of the suture strand 150 are typically set in groove 108 and trimmed as indicated by the dashed line 162 (as similarly shown in FIG. 5B.)

Finally, the strand material may be glued into place if adhesive use is desired. Alternatively, the suture (or other) strand material may be glued first and then trimmed.

Further steps in preparing a useable device may include loading it into a sheath as shown in FIG. 6, followed by packaging, sterilizing, etc. To prepare the device or system for deployment, the foot is rotated into alignment with the elongate body. The foot 100 may be setup in axial alignment with body 10 so that either end 124 or 126 is leading or most distal.

For use, the construct may be held axially aligned in such a position in connection with a releasable Beath-type needle. A simpler option is to utilize a sheath 170 as previously described in connection with FIG. 6.

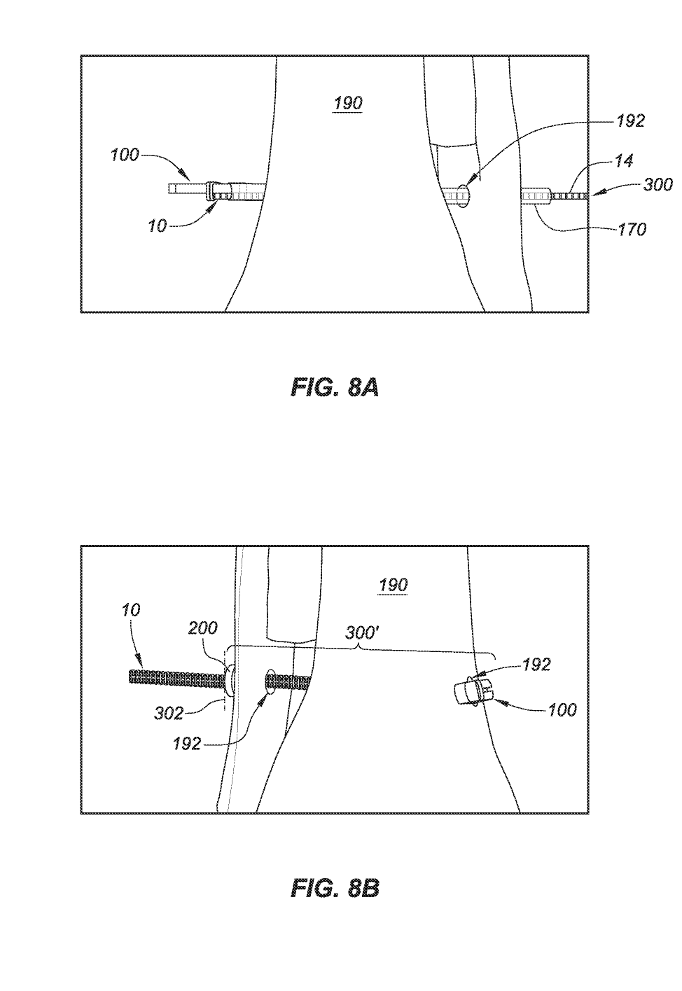

Such a device or system 300 is also shown in FIG. 8A, together with a SAWBONES model 190. For insertion through a hole 192 drilled through bone(s), the sheath adds little size to the necessary deployment hole diameter. After device 300 insertion and sheath 170 withdrawal (or partial withdrawal at least so far as to expose foot 100), the foot can be manipulated to catch and turn to its deployed position as shown in FIG. 8B. Such manipulation may be accomplished by re-advancing the sheath 170 while gripping the proximal end 14 of the elongate body 10 or otherwise.

After optional sheath removal or trimming if implantation as part of system 300′ is desired, an anchoring head 200 is applied to the elongate body 10. The example head 200 shown in FIG. 8B is secured by one-way advancement over elongate body 10, with the elongate body pulled through head 200 to set the body 10 to a desired tension. An equal amount of compression is thereby applied to the subject anatomy by the foot 100 and head 200 anchor members in the overall system.

Note that the orientation of elongate body 10 may be set in the direction shown in FIG. 8A or as in FIG. 8B, or otherwise. Naturally, the selection will determine the angular orientation of the foot 100 as well. In any case, foot 100 anchors the device against bone on one side of device 300′ and head 200 anchors it on the other side.

Finally, a proximal end of the elongate body 10 is trimmed off (advantageously flush) as indicated by the dashed line 302. This may be accomplished with flush-cut nippers or using a cutter that may be integrated in a custom tensioning and/or cutting tool.

Variations

In addition to the embodiments disclosed above, still more variations are within the scope of this description. Indeed, other methods of use applicable to the subject devices or systems are presented in FIGS. 8-15 of the above-referenced patent applications already incorporated herein by reference.

The subject methods, including methods of use and/or manufacture, may be carried out in any order of the events which is logically possible, as well as any recited order of events. Medical methods may include any of a hospital staffs activities associated with device provision, implant introduction, positioning and/or re-positioning, and surgical access and/or closure.

Furthermore, where a range of values is provided, it is understood that every intervening value, between the upper and lower limit of that range and any other stated or intervening value in the stated range is encompassed within the invention. Also, it is contemplated that any optional feature of the inventive variations described may be set forth and claimed independently, or in combination with any one or more of the features described herein. Moreover, no limitations from the specification are intended to be read into any claims, unless those limitations are expressly included in the claims.

As used herein and in the appended claims, the singular forms “a”, “an”, and “the” include plural referents unless the context clearly dictates otherwise. In other words, use of the articles allow for “at least one” of the subject items in the description above as well as the claims below. The claims may exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The subject matter described herein and in the accompanying figures is done so with sufficient detail and clarity to permit the inclusion of claims, at any time, in means-plus-function format pursuant to 35 U.S.C. Section 112, Part (f). However, a claim is to be interpreted as invoking this means-plus-function format only if the phrase “means for” is explicitly recited in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, acts, steps, or elements of the embodiments may be recited in or added to the claims, as well as negative limitations that define the inventive scope of the claims by features, functions, acts, steps, or elements that are not within that scope.

1. A medical device comprising:
an elongate body including a proximal end and a distal end;
an anchor or anchoring foot including a first end, a second end and opposing side grooves there-between; and
at least one strand, the at least one strand received through the distal end of the elongate member and around the foot to rotatably attach the foot to the elongate body.

2. The medical device of claim 1, wherein the foot includes no through-holes.

3. The medical device of claim 1, including only one strand.

4. The medical device of claim 3, wherein the strand is configured as a loop.

5. The medical device of claim 4, wherein the loop includes a splice section.

6. The medical device of claim 5, wherein the splice section is fixed with adhesive.

7. The medical device of claim 5, wherein the splice section is received in a recess formed in a distal surface of the foot.

8. The medical device of claim 7, wherein the splice section is fixed in place with adhesive.

9. The medical device of claim 1, the side groves located midway along the foot.

10. The medical device of claim 9, wherein the side groves are shaped as half cylinders.

11. The medical device of claim 1, wherein the side groves are diametrically opposed.

12. The medical device of claim 1, wherein the foot has a shape selected from oval, racetrack and rectangular forms.

13. The medical device of claim 1, wherein the foot comprises PEEK polymer material.

14. The medical device of claim 1, further comprising an anchor head configured to receive the elongate body.

15. The medical device of claim 14, wherein the anchor head includes at least one tooth configured for one-way advancement and locking with the elongate body.

16. A component of a medical device, the component comprising:
a foot having a width, a thickness and a length, wherein the length is between at a first end and a second end of the foot; and
a first groove and a second groove formed in the foot, wherein the grooves are positioned across the width from one another, span the thickness, and are located along the length between the first and second ends.

17. The foot of claim 16, wherein the grooves are located on a midline of the foot, the midline across the width and between the first and second ends

18. The foot of claim 17, further comprising a groove in a side of the foot located between the width and length, the groove running from about the midline to adjacent one of the ends.

19. The foot of claim 16, wherein the foot length is about 12.5 mm, the width is about 3 mm and the thickness is about 1.5 mm.

20. The foot of claim 16, wherein the foot comprises PEEK polymer material.

21-31. (canceled)
